(19) United States
(12) Patent Application Publication
(10) Pub. No.: US 2019/0392919 A1
(43) Pub. Date: Dec. 26, 2019

(54) BIOREACHABLE PREDICTION TOOL

(71) Applicant: Zymergen Inc., Emeryville, CA (US)

(72) Inventors: Erik Jedediah Dean, Lafayette, CA (US); Alexander G. Shearer, San Francisco, CA (US); Michelle L. Wynn, Alameda, CA (US)

(73) Assignee: Zymergen Inc., Emeryville, CA (US)

(21) Appl. No.: 16/538,622

(22) Filed: Aug. 12, 2019

Related U.S. Application Data
(63) Continuation of application No. PCT/US2018/018234, filed on Feb. 14, 2018.
(60) Provisional application No. 62/459,558, filed on Feb. 15, 2017.

(51) Int. Cl. G16B 5/00; G16B 20/00

(52) U.S. Cl. CPC G16B 5/00; G16B 20/00

(57) ABSTRACT

Systems and methods for predicting the viability of producing target molecules in a host organism are provided. A starting metabolite set for the host and a reaction set are obtained. Included in a filtered reaction set are reactions that are indicated as catalyzed by one or more corresponding catalysts that are themselves indicated as likely available to catalyze the reactions in the host organism. In each processing step, pursuant to the reactions of the filtered reaction set, data representing the starting metabolites and metabolites generated in previous processing steps is processed, to generate data representing one or more viable target molecules.

### CROSS-REFERENCE TO RELATED APPLICATIONS

This application claims priority to U.S. Provisional Application No. 62/459,558, filed Feb. 15, 2017, which is incorporated by reference in its entirety herein.

### GOVERNMENT INTEREST STATEMENT

This invention was made with United States Government support under Agreement No. HR001 1-15-9-0014, awarded by DARPA. The Government has certain rights in the invention.

### FIELD

The disclosure relates generally to methods which improve genetic engineering of microorganisms, and, in particular, to methods which improve genetic engineering of microorganisms by identifying the set of molecules that can be produced in a particular microorganism without extensive manual intervention, thereby facilitating processes such as host selection and pathway engineering.

### BACKGROUND

Chemists and material scientists employ synthetic biology to modify the genome of host organisms (e.g., bacteria, yeast or fungi) to produce desired chemicals. However, there is a limit to which chemicals can be produced as part of the biomass in a microorganism. Generally, one is faced with the problem of determining the largest possible pool of chemicals that may be generated through genome modification without requiring extensive manual intervention. Such chemicals shall be referred to herein as “bioreachable” chemicals, molecules, or metabolites.

The current state of the art in biological chemical generation may be largely broken down into two categories:

1) There is an understood target molecule or metabolic pathway—chemical production focuses on this specific pathway, and tries to force the chemicals in this pathway to be useful.

2) There is an attempt to computationally predict which molecules can be made by using a subset of known metabolic reactions and doing simple tracing through them.

These approaches are prone to errors, some resulting in very high false positive rates. The needs exists for methods to more accurately predict the chemicals that a host organism is able to biologically generate, given a set of constraints.

### SUMMARY OF THE DISCLOSURE

The present disclosure provides a bioreachable prediction tool for predicting viable target molecules in a manner that overcomes the disadvantages of conventional techniques. In particular, the bioreachable prediction tool of the present disclosure predicts viable target molecules that are specific to a specified host organism.

The bioreachable prediction tool of embodiments of the disclosure obtains a starting metabolite set specifying starting metabolites for the host organism. In embodiments, the starting metabolite set specifies core metabolites, the core metabolites including metabolites indicated by at least one database as produced by an un-engineered host under specified conditions. In embodiments, the host has not been subjected to genomic modification.

In embodiments, the bioreachable prediction tool obtains a starting reaction set specifying reactions. In embodiments, the tool includes in a filtered reaction set one or more reactions from the starting reaction set that are indicated in at least one database as catalyzed by one or more corresponding catalysts, e.g., enzymes, that are themselves indicated as likely available to catalyze the one or more reactions that may take place in the host organism.

A catalyst is likely “available to catalyze” a reaction in a host organism if the bioreachable prediction tool determines information from, e.g., public or proprietary databases, indicating that the catalyst may be introduced into the host either by engineering the catalyst into the host (e.g., by modifying the host genome) or via uptake of the catalyst from the growth medium in which the host is grown.

More specifically, this disclosure refers to a part, such as a catalyst, as being “engineered” into a host organism when the genome of the host organism is modified (e.g., via insertion, deletion, replacement) so that the host organism produces the catalyst (e.g., an enzyme protein). If, however, the part itself comprises genetic material (e.g. a nucleic acid sequence acting as an enzyme), the “engineering” of that part into the host organism refers to modifying the host genome to embody that part itself.

A part is likely “available to be engineered” into the host organism if the bioreachable prediction tool determines information indicating that the part can be engineered into the host. For example, according to embodiments, the tool would determine information indicating that an enzyme is likely available to be engineered into a host if public or proprietary databases accessed by the tool indicate (e.g., via annotation) that the enzyme is indicated as corresponding to a known amino acid sequence. If the amino acid sequence is known, then skilled artisans would be able to derive the corresponding genetic sequence used to code the amino acid sequence, and modify the host genome accordingly.

In this context and in the claims, “likely” means more probable than not, i.e., having a greater than 50% likelihood.

In each processing step of one or more processing steps, the bioreachable prediction tool processes, pursuant to the one or more reactions of the filtered reaction set, data representing the starting metabolites and metabolites generated in previous processing steps, to generate data representing one or more viable target molecules. The tool provides, as output, data representing the one or more viable target molecules.

In embodiments, the bioreachable prediction tool determines a degree of confidence as to whether a corresponding catalyst is available to catalyze the one or more reactions in the host organism, e.g., available to be engineered into the host organism to catalyze the one or more reactions. The degree of confidence may include, for example, at least a first degree of confidence or a second degree of confidence higher than the first degree of confidence. The tool may include, in the filtered reaction set, one or more reactions from the starting reaction set that are indicated in at least one database as catalyzed by one or more corresponding catalysts that are themselves determined to be available, with the second degree of confidence, to catalyze the one or more reactions in the host organism, e.g., determined to be available, with the second degree of confidence, for engineering into the host organism to catalyze the one or more reactions.

In embodiments of the disclosure, the bioreachable prediction tool generates an indication of the difficulty of producing one or more of the viable target molecules. The indication of difficulty may be based upon thermodynamic properties, reaction pathway length for the one or more viable target molecules, or a degree of confidence as to whether a catalyst is available to catalyze one or more corresponding reactions along one or more first reaction pathways to one or more of the viable target molecules.

In embodiments of the disclosure, after generating data representing one or more viable target molecules in a particular processing step and before the next processing step, the bioreachable prediction tool removes from the filtered reaction set any reactions associated with generating the data representing one or more viable target molecules in the particular processing step.

In embodiments, the tool generates a record of one or more reaction pathways (i.e., pedigrees) leading to each viable target molecule. In embodiments, generating a record comprises not including in the record reaction pathways from ubiquitous metabolites. In embodiments, the tool generates a record of the step in which data representing a viable target molecule is generated. In embodiments, the tool generates a record of the shortest reaction pathway from the starting metabolite set to each viable target molecule.

Instead of determining viable target molecules given a single host organism, it may be desired to identify one or more host organisms in which to produce a given viable target molecule. For example, a customer may ask the user of the tool to determine the optimum host organism within a plurality of hosts in which to produce the target molecule. In embodiments, the bioreachable prediction tool is run for a plurality of host organisms, and generates data representing one or more viable target molecules, according to any of the methods described herein, for each host organism of the plurality of host organisms. In such embodiments, for a given viable target molecule, the tool determines at least one of the plurality of host organisms that satisfies at least one criterion, such as a given predicted yield of the viable target molecule produced by a given host organism or a given number of processing steps predicted as necessary to produce the given viable target molecule in a given host organism. The tool provides, as output, data representing the host organisms determined to satisfy the at least one criterion.

As described for embodiments above, the tool may generate a record, including, e.g, thermodynamic properties, of one or more reaction pathways (i.e., pedigrees) leading to each target molecule produced by each host organism. Based on the above embodiments running the tool for the plurality of host organisms, the tool may store associations between host organisms, target molecules, and pedigrees in a database as a library, which may include annotations specifying parameters such as yield, number of processing steps, availability of catalysts to catalyze reactions in the reaction pathways, etc.

In embodiments, if the tool has access to such a library, the tool need not be run to identify multiple host organisms in which to produce a given viable target molecule. Instead, in such embodiments, the tool may use the pedigrees from the library, which may include annotation data concerning associations among the hosts, target molecules, and reactions. The tool may identify at least one target host organism from among the one or more host organisms based at least in part upon evidence, from, e.g., public or proprietary databases or from the library, that all the catalysts predicted to catalyze reactions in at least one reaction pathway leading to production of the target molecule in the at least one target host organism are likely available to catalyze all such reactions. In embodiments, the tool may determine target hosts based upon the target hosts requiring less than a threshold number of reaction steps within the reaction pathways that are predicted as necessary to produce the target molecule.

Some reaction enzymes may not have a known associated amino acid sequence or genetic sequence (“orphan enzymes”). In such cases, the tool may bioprospect the orphan enzymes to predict their amino acid sequences, and, ultimately, their genetic sequences, so that the newly-sequenced enzymes may be engineered into the host organism to catalyze one or more reactions. The tool may include the reactions corresponding to the newly-sequenced enzymes as members of the filtered reaction data.

In embodiments, the bioreachable prediction tool provides to a “factory,” e.g, a gene manufacturing system, an indication of one or more genetic sequences associated with one or more reactions in a reaction pathway leading to a viable target molecule. In embodiments, the gene manufacturing system embodies the indicated genetic sequences into the genome of the host, to thereby produce an engineered genome for manufacture of the target molecule. In embodiments, the tool provides to the factory an indication of one or more catalysts for the factory to introduce the one or more catalysts into the growth medium of the host organism for production of the target molecule.

In embodiments, the bioreachable prediction tool includes, in the filtered reaction set, reactions from the starting reaction set based at least in part upon whether the one or more reactions are spontaneous, based at least in part upon their directionality, based at least in part upon whether the one or more reactions are transport reactions, or based at least in part upon whether the one or more reactions generate a halogen compound.

In embodiments of the disclosure, the bioreachable prediction tool obtains a starting metabolite set specifying starting metabolites for the host organism, and obtains a starting reaction set specifying reactions specific to the host. In embodiments of the disclosure, the bioreachable prediction tool includes in a filtered reaction set one or more reactions that are indicated as spontaneous in at least one database. In each processing step of one or more processing steps, the tool processes, pursuant to the one or more reactions of the filtered reaction set, data representing the starting metabolites and any metabolites generated in previous processing steps, to generate data representing one or more viable target molecules in each step. In embodiments, the tool provides, as output, data representing the one or more viable target molecules.

### BRIEF DESCRIPTION OF THE DRAWINGS

FIG. 1 illustrates a system for implementing a bioreachable prediction tool according to embodiments of the disclosure.

FIG. 2 is a flow diagram illustrating operation of a bioreachable prediction tool according to embodiments of the disclosure.

FIG. 3 illustrates pseudocode for implementing strict and relaxed enzyme sequence searches according to embodiments of the disclosure.

FIG. 4 illustrates an example of a report that may be generated by the bioreachable prediction tool of embodiments of the disclosure.

FIG. 5 illustrates a hypothetical example of a report of reaction pedigree tracking that may be generated by the bioreachable prediction tool of embodiments of the disclosure.

FIG. 6 illustrates a cloud computing environment according to embodiments of the disclosure.

FIG. 7 illustrates an example of a computer system that may be used to execute instructions stored in a non-transitory computer readable medium (e.g., memory) in accordance with embodiments of the disclosure.

FIG. 8 illustrates an example of a single pathway of the type that may be generated by the biroeachable prediction tool of embodiments of the disclosure. In this example, the molecule tyramine was predicted to be reachable by addition of a single enzymatic step to a host organism. This pathway has been reduced to practice and engineered into host organisms to produce tyramine. This pathway's evaluation score is appended at the end of the reaction diagram.

FIG. 9 illustrates an example of two distinct pathways of the type that may be generated by the bioreachable prediction tool of embodiments of the disclosure. In this example, both pathways were identified by the bioreachable prediction tool as being able to generate the bioreachable molecule (S)-2,3,4,5-tetrahydrodipicolinate (TUDP). The two pathways differ by their use of reducing equivalent types (NADH versus NADPH). One of these pathways has been reduced to practice and engineered into host organisms to produce TUDP. Each pathway's evaluation score is appended at the end of the reaction diagram.

FIG. 10 illustrates an example of a more complex multi-pathway prediction of the type that may be generated by the bioreachable prediction tool of embodiments of the disclosure. Each pathway's evaluation score is appended at the end of the reaction diagram.

FIGS. 11A and 11B together illustrates an example of a scoring breakdown that may be generated by the bioreachable prediction tool of embodiments of the disclosure. (FIG. 11B appends to the bottom of FIG. 11A.) In this case, the evaluation data shown was generated during the process of predicting pathways to the molecule (S)-2,3,4,5-tetrahydrodipicolinate (THDP).

### DETAILED DESCRIPTION

The present description is made with reference to the accompanying drawings, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

The inventors have recognized that conventional methods for predicting viable target molecules suffer from the following obstacles:

1) Lack of biological parts. This is the single biggest cause of false positive predictions about chemicals that can be biologically generated. Some conventional methods employ existing reaction databases to step through all known metabolic reactions from a feedstock like glucose, and assume all paths can be engineered. However, many reactions do not correspond to a genetic part that can be engineered into a host organism. Typically, reactions are catalyzed by enzymes. Reactions in existing databases may be well-characterized according to their catalyzing enzymes, but many of those enzymes have not had their amino acids sequenced, meaning that there is no established correlation between the enzyme and an associated gene sequence. Without a gene sequence, a host genome cannot be modified to produce the required enzyme. In fact, approximately 25-50% of well-characterized enzymatic reactions have no known associated gene sequence, and thus those enzymes are not useful as biological parts for engineering purposes. The percentage of gene-absent reactions in overall biological databases is likely even higher, since these databases include many reactions that are not well-characterized. The inventors note that, in some cases, catalysts other than enzymes, such as enzyme-nanoparticle conjugates, may be employed. See, e.g., Vertgel A A, et al., Enzyme-nanoparticle conjugates for biomedical applications, Methods Mol. Bio. 2011; 679: 165-82; Johnson P A, et al., Enzyme nanoparticle fabrication: magnetic nanoparticle synthesis and enzyme immobilization, Methods Mol. Biol. 2011; 679: 183-91, all of which are incorporated by reference in their entirety herein. In those cases, parts required to engineer those catalysts into a host organism may or may not be known.

2) Incorrect pathway tracing. Many attempted solutions attempt to trace pathways arbitrarily between molecules. This can lead to a failure to properly track the creation of the target molecule's carbon skeleton. To cite a common example, a path might be traced from glutamine into a reaction that generates a target molecule, and then glutamine would be cited as a part of the path to creating that target molecule. However, in most cases, glutamine is donating a nitrogen group and no carbon, so this tracing is misleading and does not indicate that the target molecule can be made (other errors include tracing connections though other ubiquitous molecules such as ATP or inorganic molecules such as water). These types of pathway tracing errors also lead to an unusably large number of predicted pathways (as if a mapping application allowed all possible street routes through San Francisco, instead of the two to three most direct and useful paths).

3) Assuming bidirectional reactions. Another significant source of errors is failure to account for the thermodynamics/direction of reactions. Thermodynamics dictates that some reactions can only run in one direction. A reaction that only degrades molecule A to molecule B, however, is often predicted by conventional means to run in either direction, so it would be incorrectly predicted that molecule A can be synthesized from B. As a particular example, some bacteria breakdown halogenated compounds such as organochlorides, but cannot run in reverse to create halogenated compounds. Because many biological reactions are dramatically favored to run in only one direction, failure to account for reaction directionality would also create a false positive prediction.

4) Other errors. Not every host can be engineered to produce every target molecule, or be engineered to produce every target molecule with the same set of modifications or likelihood of success, because not all hosts maintain the same set of metabolic pathways.

The bioreachable prediction tool (BPT) of embodiments of the disclosure overcomes the limitations of conventional methods. The BPT of embodiments of the disclosure may describe, in a target-agnostic fashion, every chemical that likely can be biologically generated given a set of starting constraints (e.g. particular host organism, number of reaction steps, whether only reactions with gene-sequenced enzymes allowed). This creates a “bioreachable list,” a list of viable target chemicals. These target chemicals and their associated structures can be provided to professional chemists, who can review the chemical utility of the molecules without having to consider the biology required to create them. After particular bioreachable target chemicals are selected, their formulas and reaction pathways may be provided to a gene manufacturing system to modify the gene sequence of the host organism to produce the selected target molecules.

### System Design

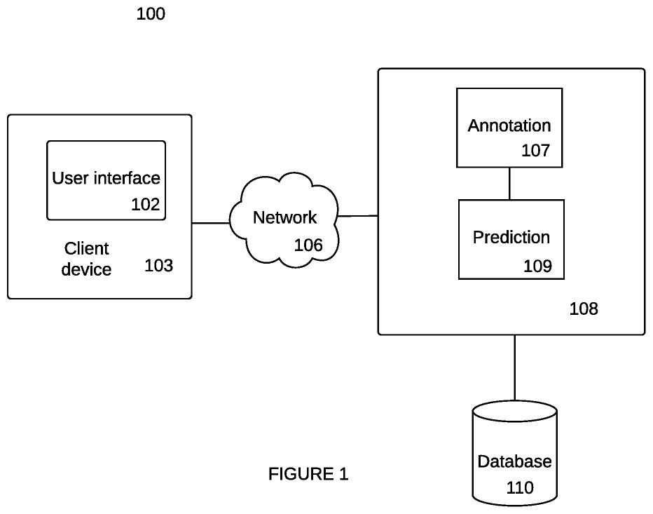

FIG. 1 illustrates a distributed system 100 of embodiments of the disclosure. A user interface 102 includes a client-side interface such as a text editor or a graphical user interface (GUI). The user interface 102 may reside at a client-side computing device 103, such as a laptop or desktop computer. The client-side computing device 103 is coupled to one or more servers 108 through a network 106, such as the Internet.

The server(s) 108 are coupled locally or remotely to one or more databases 110, which may include one or more corpora of molecule, reaction, and sequence data. The reaction data may represent the set of all known metabolic reactions. In embodiments, the reaction data is universal, i.e., not host-specific.

The molecule data includes data on metabolites—reactants involved in the reactions contained in the reaction data as either substrates or products. In embodiments, the data on metabolites includes data on host-specific metabolites, such as core metabolites, known in the art to be produced in particular host microorganisms. In some embodiments, some core metabolites were determined to be produced by a particular host through empirical evidence gathered by the inventors. These host-specific metabolite sets were identified through various methods such as metabolomics analysis of the host organism or by identifying enzyme-coding genes that are essential under certain growth conditions, and inferring the presence of metabolites produced by the enzymes coded by those genes. The molecule data may be tagged with annotations representing many features, such as host organism, growth medium characteristics, and whether a molecule is a core metabolite, a precursor, ubiquitous, or inorganic.

The database(s) 110, e.g., UniProt, may also include data on whether a catalyst may be introduced into a host organism via uptake of the catalyst from a growth medium in which the host is grown.

The sequence data may include data for the reaction annotation engine 107 to annotate reactions in the reaction data set as to whether a reaction is likely known to correspond to sequences, e.g., enzyme or genetic sequences, for engineering the reaction into a host organism. For example, the sequence data may include data for annotating reactions in the reaction data as to whether a reaction is catalyzed by an enzyme for which the corresponding amino acid sequence is likely known. If so, then, through methods known in the art, a genetic sequence for coding the enzyme can be determined. In embodiments, for purposes of determining bioreachable target molecules, the reaction annotation engine 107 does not need to know the sequence data itself, but rather only whether a sequence is likely known to exist for the catalyst. The reaction annotation engine 107, described below, may compile the sequence data from databases such as UniProt, which include sequence data for enzymes that catalyze reactions indicated as having associated coding sequences.

In embodiments, the server(s) 108 includes a reaction annotation engine 107 and a bioreachable prediction engine 109, which together form the bioreachable prediction tool of embodiments of the disclosure. Alternatively, the software and associated hardware for the annotation engine 107, the prediction engine 109, or both may reside locally at the client 103 instead of at the server(s) 108, or be distributed between both client 103 and server(s) 108. The database(s) 110 may include public databases such as UniProt, PDB, Brenda, BKMR, and MNXref, as well as custom databases generated by the user or others, e.g., databases including molecules and reactions generated via synthetic biology experiments performed by the user or third-party contributors. The database(s) 110 may be local or remote with respect to the client 103 or distributed both locally and remotely. In some embodiments, the annotation engine 107 may run as a cloud-based service, and the prediction engine 109 may run locally on the client device 103. In embodiments, data for use by any locally resident engines may be stored in memory on the client device 103.

### System Operation

Obtaining the starting metabolite lists and starting reaction data sets

Inputs to the bioreachable prediction process include information such as starting metabolite list, starting reaction list, host organism, and baseline conditions, such as fuel level for the host (e.g., minimal or rich growth medium) and environmental conditions such as temperature. The annotation engine 107 may assemble metabolite and reaction data along with associated annotations from the database(s) 110.

Through the user interface 102, a user may specify the database(s) 110 from which to obtain information for the starting metabolite and reaction lists. For example, reactions and host-specific metabolites may be obtained from public databases such as KEGG, Uniprot, BKMR, and MNXref. (Those skilled in the art will recognize from the context of the discussion that references in this specification and the claims to “metabolites,” “reactions,” and the like may in many instances in fact refer to data representing those physical objects or processes and not the physical objects or processes themselves.)

### Starting Metabolite List

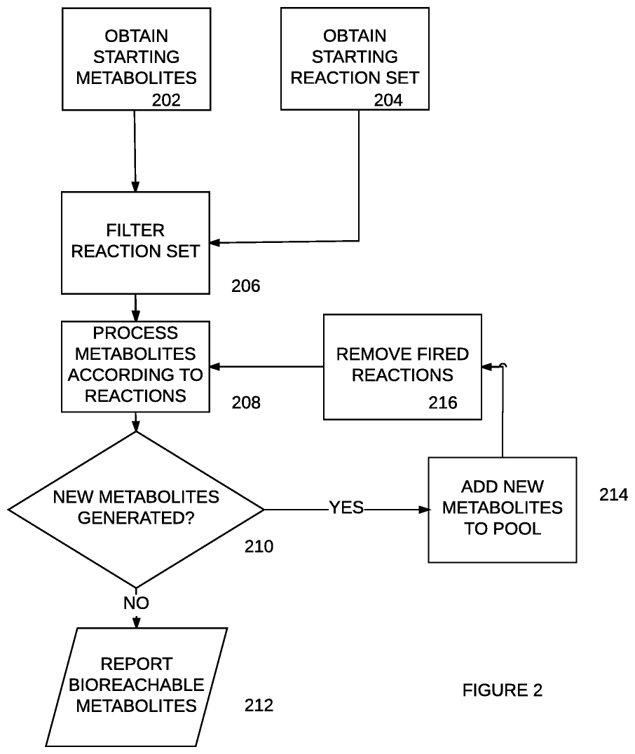

Referring to FIG. 2, in embodiments the reaction annotation engine 107 obtains or itself aggregates from the database(s) 110 a host-specific starting metabolite file comprising a list of chemical compounds (starting, intermediate, and final products) that are expected to be present during the growth of the host organism at a particular time or during a particular time interval under given growth conditions (202). The default growth condition may be a minimal growth medium, because this is the most conservative approach for selecting the starting metabolites. In embodiments, the reaction annotation engine 107 may provide the metabolite file as a starting metabolite list to the prediction engine 109.

In embodiments, the reaction annotation engine 107 may determine or template (off of similar microbes) the starting metabolites based on growth data for the host organism or for a similar organism. This approach is similar to approaches used to annotate the genomes of microbes in systems such as the RAST system, or to predict metabolic pathways in the BioCyc database collection. This approach uses the genome annotation for a given host organism to make a best guess at which metabolic pathways are present, and then assumes the presence of all the constituent reactions, and their metabolites, in those pathways. In the case of BioCyc databases, the existing genome annotation is used to identify the putative presence of individual enzymes (and thus their reactions). A rule-based system is then used to infer the presence of entire metabolic pathways based on the presence of (some of) their substituent reactions.

Having a starting metabolite list that is specific to the host organism is a distinguishing starting point for embodiments of the disclosure. Whereas other conventional approaches make generic predictions about targets that can be made, this customizable step of embodiments of the disclosure avoids the problem of making incorrect predictions about which target molecules can be made (or how they can be made) due to differences in the biology of host organisms.

In embodiments, the user may instruct the reaction annotation engine 107 to retrieve the starting metabolites from existing databases or datasets, such as MNXref, KEGG or BKMR, based upon querying the databases or datasets with parameters such as host organism and growth medium, and, in some embodiments, via cross-indexing those databases with relevant model organism databases or other indications of the presence of specific metabolites. So far, for particular industrial hosts the assignees have created typical starting metabolite files on the order of 200-300 metabolites. As noted above, data objects representing metabolites in the public databases and the lists formed by the annotation engine 107 may include annotations including metadata such as host organism, growth medium type, and whether the metabolite is a core metabolite, a precursor, inorganic, or ubiquitous.

Core metabolites are the starting (e.g., substrate), intermediate and final metabolites natively found in a genetically-unmodified microorganism for given baseline conditions, such as the richness of the growth medium. Each core metabolite (e.g., amino acid) in the biomass of a microorganism like E. coli may be generated in the cell's core metabolism from one of eleven precursor metabolites, and may be fundamentally generated from whatever carbon input is provided to the genetically-unmodified organism. In embodiments, the user may select a starting metabolite set of select core compounds tagged with their precursor dependencies from databases such as MNXref, KEGG, ChEBI, Reactome, or others.

As their name suggests, inorganic metabolites, such as ammonium, do not include carbon, and thus cannot contribute a carbon atom to new products of the metabolism. Accordingly, the reaction annotation engine 107 may exclude inorganic metabolites from the starting metabolite set.

Some metabolites are ubiquitous, i.e., they are found in many reactions. They include molecules like ATP and NADP. Typically, ubiquitous molecules do not contribute carbon to the target product, and thus would not be part of any metabolic pathway to the target. Accordingly, the reaction annotation engine 107 may exclude ubiquitous metabolites from the starting metabolite set. Ubiquitous molecules can be manually designated in annotations based on expert evaluation or identified by determining what molecules participate in reactions beyond a particular threshold number. One heuristic flags all molecules that appear in the reaction set at numbers greater than the size of a typical core metabolite input (e.g., 300). For example, in one data set ATP appears in 2,415 of approximately 31,000 reactions, NADH appears in 2,000 reactions, and NADPH appears in 3,107 reactions, which places them above the core metabolite count and earns them all the “ubiquitous” tag.

### Starting Reaction Data Set

The reaction annotation engine 107 obtains a starting reaction data set as the basis for prediction of viable target molecules (204). The user may specify how to build the starting reaction data set, or the user may instruct the annotation engine 107 to obtain the data directly from a public database 110 or a proprietary database 110, such as a custom database previously created by the user or others. In one embodiment, the annotation engine 107 may import the full reaction set (approximately 30,000 reactions) from the MetaNetx reaction namespace (MNX) of MNXref. In other embodiments, the annotation engine 107 may import and merge the reaction sets (approximately 22,000 total reactions) from MetaCyc and KEGG, or other public or private databases.

In embodiments, the reaction annotation engine 107 may build the starting reaction data set by selectively aggregating the information obtained from the database(s) 110. For example, BKMR provides information whether a reaction is spontaneous. The annotation engine 107 may use known mappings to map BKMR reaction IDs to IDs in MNXref for corresponding reactions. In other examples, KEGG or MetaCyc and their IDs may be employed instead of BKMR and its IDs. Using this association, the reaction annotation engine 107 may then create a custom reaction list in database(s) 110 using the existing annotations from MNXref (e.g., core, ubiquitous), along with a corresponding spontaneous reaction tag from BKMR. Similarly, through mapping corresponding IDs, the annotation engine 107 may associate reactions in MNXref with annotations in UniProt to obtain tags for whether a reaction is a transport reaction or whether a reaction substrate or product contains a halogen, and incorporate those tags into the annotations for the reaction in the custom reaction list in database(s) 110. (Identifying halogenated compounds is a heuristic for identifying reactions that run in the wrong direction, since most halogen-related reactions concern breaking down a chemical.)

Along these lines, the reaction annotation engine 107 may use associated IDs across databases to aggregate data from the databases to build a database 110 storing starting reaction sets with custom annotations, such as whether the reaction is spontaneous, runs in only one direction due to thermodynamics, contains a halogen (related to determining directionality), contains a ubiquitous metabolite, is a transport reaction, is unbalanced (that is, the two sides of the chemical reaction do not maintain elemental balance, suggesting the reaction is improperly written in the source database and should be ignored), is incompletely characterized in available databases, is associated with enzymes tagged with an indicator that the enzyme is associated with a known amino acid sequence or genetic sequence coding the enzyme, or is catalyzed by source enzymes likely to have transmembrane domains, among other tags. Through the annotation engine 107, the user may thus assign annotations to all of the approximately 30,000 reactions in the MNXref database, for example. As described below, the user may then configure criteria to filter this master file into individual lists for each annotation feature or any combination thereof.

### Bioreachable Molecule Prediction

Referring to the flow diagram of FIG. 2, the following describes examples of the operation of the prediction engine 109 of embodiments of the disclosure. The prediction engine 109 predicts which chemicals can be created via, e.g., genetic engineering, in an arbitrarily selected host organism. The prediction engine 109 may take as inputs a starting metabolite file, a starting reaction data set, and a sequence database. The sequence database may store the amino acid sequences for catalytic compounds (such as enzymes), or the genetic sequences that encode catalytic compounds. In embodiments, the BPT of embodiments of the disclosure uses the sequence database to determine the presence or absence of an amino acid sequence or genetic sequence for each reaction. In such embodiments, the sequence database need not include the sequences themselves, as long as the catalysts are tagged as having an enzyme or genetic part available or not. Along with a list of bioreachable molecules, the prediction engine 109 produces for a specified host organism “pedigrees” (reaction pathways) of the reactions leading to production of each reachable target molecule from the starting metabolites, e.g., the host's core metabolites in some embodiments.

In particular, the predictions can be tuned based on a number of parameters, such as likely availability of catalysts to catalyze reactions, (e.g., likely availability of genetic parts to be engineered into the host organism or likely availability of catalysts to be introduced into the host organism via uptake from a growth medium in which the host organism is grown), maximum number of reaction steps allowed (starting from the starting metabolites), types of parts or chemical reactions to be allowed, and other selectable features. The prediction engine 109 also helps predict the approach to, and difficulty in designing target molecules by predicting the potential paths from core metabolites to each target molecule.

### Filtered Reaction Data Set

In embodiments, the prediction engine 109 creates a filtered and validated reaction data set (RDS). Using the reactions characterized by the reaction annotation engine 107, the prediction engine 109 may filter the reactions to a desired level of validation, e.g., level of confidence that a coding sequence for the reaction enzyme exists (206). This is a step in fine tuning the accuracy of the predictions, and for controlling the primary source of false positive predictions. In an example mentioned above, the inventors generated the RDS for one bioreachable list by importing and annotating the full reaction set (approximately 30,000 reactions) from the MetaNetx reaction namespace (MNX) of MNXref. A similar approach could be applied to other publicly available reaction databases such as KEGG, Reactome, and MetaCyc.

Based upon the inventors' experience, 25-50% of the reactions in the most popular public databases may not have any known associated biological parts. For example, the amino acid sequences of enzymes for catalyzing the reactions, or their accompanying genetic sequences, may be unknown. Without the enzyme sequence information, a bioreactor would not be able to perform the reactions employing those enzymes, thus rendering the reaction information useless for engineering purposes. Even if only one enzyme within a pathway lacks a known gene sequence, then the entire pathway cannot be engineered into a host.

To deal with this deficiency, the prediction engine 109 may filter the reactions through a series of validation tests using publicly available or custom enzyme data. One public database is UniProt, which is large, open access, and reliably curated. Others include the RCSB Protein Data Bank (PDB) and GenBank In some public databases such as MNXref, UniProt, Brenda, or PDB, reactions may be tagged with an Enzyme Commission (EC) number, which is a numerical classification for enzymes based on the reactions they catalyze. Some databases, such as UniProt or PDB, store EC number tags only for reactions for which the gene sequence coding the catalyzing enzymes are known. Other databases, such as KEGG and MetaCyc, include EC numbers for enzymes for which the gene sequence is not known.

Thus, depending upon the database, an EC number may or may not indicate the existence of a known enzyme gene sequence. Approximately, 20-25% of reactions with EC numbers have no associated enzyme coding sequence. In some cases, EC numbers are used to annotate multiple specific chemical transformations (there is a one-to-many relationship between EC numbers and chemical reactions), so that the presence of an enzyme sequence associated with an EC number does not mean that every reaction associated with that EC has a valid associated sequence. Thus, the presence of an EC tag on an enzyme activity is not a reliable general indicator of the presence of a gene sequence for that enzyme, but it can be applied to certain databases to determine if a sequence is reasonably likely to be present for that enzyme. Some databases also have separate fields (e.g. the “catalytic activity” field in UniProt) that explicitly describe particular chemical reactions as known to be definitively catalyzed by a given amino acid sequence (thus having a known genetic sequence for coding the enzyme catalyst). Such reactions are referred to herein as annotated as “definitively sequenced.”

The prediction engine 109 may determine a degree of confidence as to whether a catalyst is available to catalyze a reaction in the host organism (e.g., available to be engineered into the host organism to catalyze the reaction). For example, based on the differences in certainty that enzyme coding sequences are known, the prediction engine 109 may execute, in some embodiments, a “strict” search or a “relaxed” search for enzyme coding sequences against annotations in the reaction data set. For a strict search, the prediction engine 109 may select, for example, only reactions annotated as being definitively sequenced.

For a relaxed search, the prediction engine 109 may select, for example, reactions annotated as having an EC number that is associated with known enzyme coding sequences or (Boolean non-exclusive OR) reactions that are annotated as “definitively sequenced” in the sequence database, from annotations derived from databases such as MetaCyc. The prediction engine 109 records whether any gene or amino acid sequences are found for the reactions, for either level of confidence. For example, the prediction engine 109 may annotate the reaction with a tag indicating that it satisfies the relaxed search, but not the strict search.

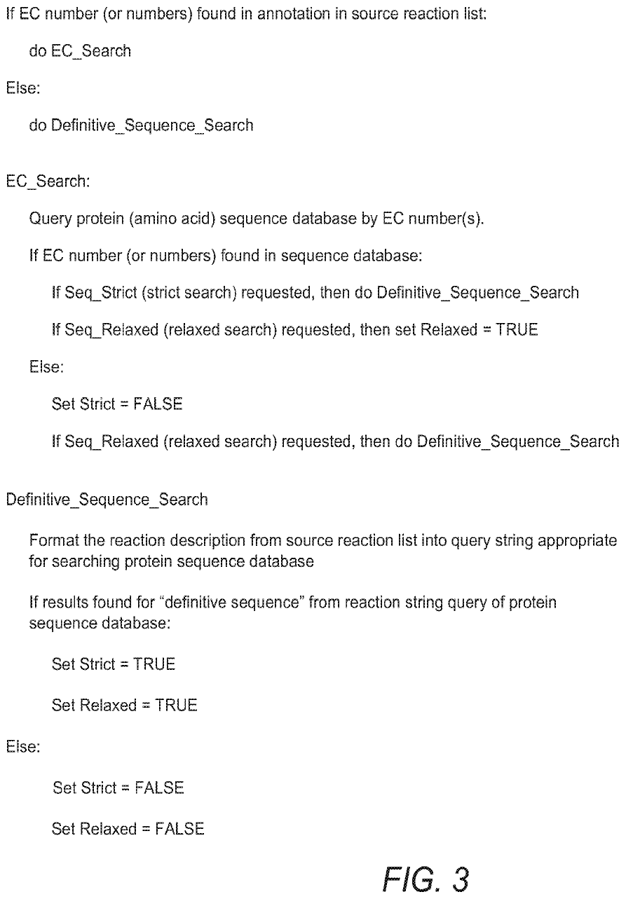

FIG. 3 illustrates exemplary pseudocode for implementing strict and relaxed enzyme sequence searches against databases, such as MNXref and UniProt, according to embodiments of the disclosure. The pseudocode describes the logic used by a heuristic for determining whether a sequence exists for an enzyme. This embodiment provides four levels of confidence. The code shows first determining whether the reaction data set annotations include at least one EC number. If so, then the code calls for searching the sequence database for EC numbers. If a strict search is being conducted, then the code calls for searching the sequence database for reactions that are definitively sequenced. If a relaxed search is being conducted, then the code sets the Relaxed annotation tag for the reactions having associated EC numbers to TRUE.

If the initial step determines that the reaction data set annotations (a) do not include an EC number or (b) (as mentioned above) the EC sequence search finds an EC number in the sequence database and a strict search is being conducted, then the code calls for searching the sequence database for reactions that are definitively sequenced. If that search finds a reaction as definitively sequenced, then the code sets both the Strict and Relaxed annotations for that reaction as TRUE. If not, then the code sets both those annotations for that reaction as FALSE.

In sum, the output of this heuristic are two annotation tags for each reaction: Strict and Relaxed. This heuristic provides four levels of confidence, as described below:
Strict=TRUE→very high confidence a sequence exists
Strict=FALSE→moderate confidence a sequence does not exist (expect some false negatives)
Relaxed=TRUE→moderate confidence a sequence exists (expect some false postivies)
Relaxed=FALSE→very high confidence a sequence does not exist

The inventors have found that running a relaxed search results in less than a 20% false positive rate, whereas running a strict search against the catalytic activity field in UniProt results in a significant false negative rate. Thus, it may be better to en slightly on the side of a relaxed search. The “relaxed” and “strict” tags are just two potential methods of handling sequence-based filtering. The BPT is amenable to any sequence-based tagging (and thus filtering) approach, including more permissive methods such as identifying the presence of sequences with appropriate motifs for the target activity or more stringent methods such as requiring the presence of a directly-literature-supported activity-sequence link in a heavily curated database such as MetaCyc.

As an alternative or in addition to the sequence-based filtering, the prediction engine 109 may filter (i.e., select or not select) reactions based upon any combination of the annotations discussed above with respect to the annotation engine 107, such as reaction directionality, or whether a reaction is a spontaneous reaction, a transport reaction, or contains a halogen. The prediction engine 109 may perform filtering based on user configuration through the user interface 102 or default settings. In embodiments, the prediction engine 109 may apply different filters in different reaction steps along the simulated metabolic pathways. As an example of default settings, they may be: reaction has a sequence based on relaxed criteria; exclude all transport reactions; only include reactions containing halogens if the reactions have a sequence; include all spontaneous reactions regardless of the above attributes.

If a reaction is spontaneous, the reaction will occur automatically without the need to engineer the host genome to produce an enzyme to catalyze the spontaneous reaction. Since the reaction is known to occur under given conditions for a given host, the prediction engine 109 can predict that the spontaneous reaction products will be produced.

As noted above, inorganic molecules do not contribute carbon and ubiquitous molecules are unlikely to contribute carbon to target metabolites. Thus, eliminating ubiquitous and inorganic molecules from those used as starting metabolites heuristically provides a high confidence level that the prediction engine 109 will follow valid metabolic pathways in predicting viable target molecules. Accordingly, the prediction engine 109 does not treat ubiquitous or inorganic molecules as limited in a reaction. That is, they are assumed to always be available to the reactions in which they participate.

### Metabolite Prediction

Referring to FIG. 2, the prediction engine 109 may perform a stepwise simulation to predict which metabolites would be formed, given a substrate of input metabolites processed according to the reactions in the filtered RDS (208). (A chemical reaction operates on an input “substrate” (e.g., set of molecules) to produce chemical products.) The operation of the prediction engine 109 of embodiments of the disclosure may be described as follows:

Step 0: Initially, only core metabolites are present in the simulated host organism. They form the current substrate for the reactions in the next step.

Step 1: The prediction engine 109 determines whether the core metabolites from step 0 match one side of any of the chemical equations within the filtered reaction set (RDS), and whether a reaction can take place in a given direction (based on directional/thermodynamic annotation), to thereby determine which reactions would fire to produce chemicals on the other side of the reaction equation (208). The prediction engine 109 determines whether any new metabolites are produced by the fired reactions (210).

If the prediction engine 109 determines that no new metabolites have been predicted (210), then the prediction engine 109 ends the prediction process, and reports the results (212).

Conversely, if the prediction engine 109 determines that new metabolites would be formed (210), the prediction engine 109 adds the new metabolites to the substrate pool (214). The updated substrate pool now includes the core metabolites and the newly predicted metabolites from step 1.

The prediction engine 109 records the metabolites and fired reactions in each step, and also removes the fired reactions from the filtered RDS (step 216). This removal prevents the same reactions from being fired in subsequent steps, to thereby avoid a reaction and its resulting metabolite(s) from being identified as present in a subsequent step. Each reaction is simulated only once throughout all steps of the process. This comports with engineering best practices that generally focus on the shortest path (fewest number of steps) to reach a metabolite—longer pathways to the same metabolite are typically suboptimal. Along with the metabolites and reactions within each step, the prediction engine 109 records the step in which a metabolite is made (i.e., predicted to be made). That step represents the metabolic path length to generating the metabolite. Note that a metabolite may appear as a product in multiple steps if it is created via distinct reactions. This fact allows the prediction engine to identify usefully distinct pathways, where the same metabolite is reached by distinct reactions.

Step 2: The prediction engine 109 then returns to step 208 using the now updated substrate pool of metabolites as inputs to run against the filtered RDS (with fired reactions now removed) to predict whether any reactions would fire to produce new metabolites.

After multiple iterations, the pool of metabolites grows whereas the pool of available reactions shrinks. Eventually, the process may run to saturation because no more metabolites are left that can fire a reaction that remains in the filtered RDS. In experiments by the inventors, approximately 10,000 filtered reactions may result in several thousand metabolites after all iterations. Alternatively, the prediction engine 109 may be configured to specify the number of allowed reaction steps before halting the predictions and reporting the results (212). The limitation on number of reaction steps reflects real-world engineering, which would typically limit the number of cycles.

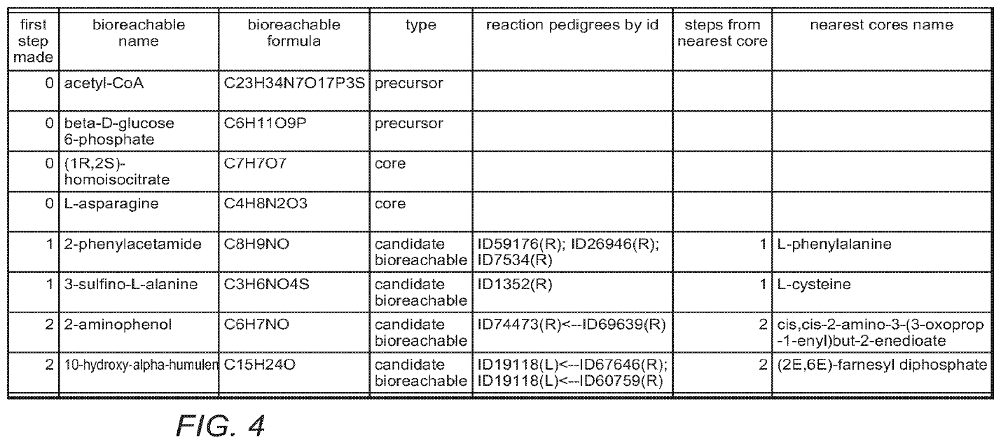

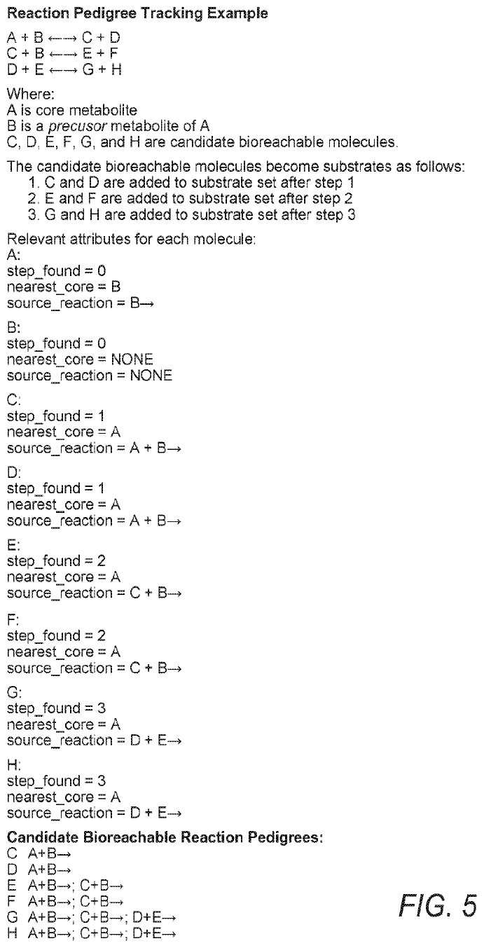

FIGS. 4 and 5 illustrate examples of reports that may be generated by the bioreachable prediction tool of embodiments of the disclosure. FIG. 4 shows, for each processing step, the metabolites generated (bioreachable name), their chemical formulas, the type of metabolite (e.g., core, precursor, candidate bioreachable produced by a reaction), the reaction pedigrees of the metabolites as denoted by a unique reaction ID such as an ID used in well-known databases (which also shows whether the left (“L”) or right (“R”) side of the reaction fired), the number of reaction steps needed from the nearest core metabolite to produce the candidate bioreachable molecule, and the name of the nearest core metabolite for each candidate bioreachable molecule. Note that the only molecules in step 0 are from the starting metabolite list (e.g., cores, precursors).

FIG. 5 illustrates a hypothetical example of reaction pedigree tracking. Stepwise the reactions are as follows:

Step 1: A+B←→C+D

Step 2: C+B←→E+F

Step 3: D+E←→G+H

The attributes in this example include: whether the metabolite generated in the step is a core; the step in which the metabolite is found; the nearest core metabolite to the generated metabolite, as measured by distance in number of steps; and the reaction pedigree denoting the chemical reaction fired to produce the metabolite. Metabolite A is a core metabolite and B is a precursor metabolite present in the biomass of the host at Step 0. Thus they have no reaction pedigree.

C and D are shown as produced in Step 1 by the reaction A+B in the reaction pedigree (source_reaction). The nearest core to both C and D is A. C and D are added to the substrate along with cores A and B .

E and F are shown as produced in Step 2 by the reaction C+B . The nearest core to both E and F is A. E and F are added to the substrate along with cores A and B and bioreachable products C and D.

G and H are shown as produced in Step 3 by the reaction D+E. The nearest core to both G and H is A.

The tool may also output the pathway (also known as the “pedigree” sequence of reactions) for each metabolite as follows:

C:A+B→

D:A+B→

E:A+B→; C+B→

F:A+B→; C+B→

G:A+B→; C+B→; D+E→

H:A+B→; C+B→; D+E→

Pathway filtering. In embodiments, given a host organism, a target molecule, and the reaction pedigrees of the pathways leading to the given target molecule, the prediction engine 109 may selectively filter the pathways to identify pathways based on given parameters, such as path length (e.g., number of reaction processing steps from starting metabolite to target molecule). The prediction engine 109 may provide, as output, data representing the identified reaction pathways.

Host organism selection. Instead of determining viable target molecules given a single host organism, it may be desired to identify one or more host organisms in which to produce a given viable target molecule. In embodiments, the prediction engine 109 generates data representing viable target molecules, according to any of the methods described above, for not just one host organism, but for a plurality of host organisms. In such embodiments, for a given viable target molecule, the prediction engine 109 determines at least one of the plurality of host organisms that satisfies at least one criterion. For example, using the reaction pedigree data, the prediction engine 109 may select a host organism based upon the number of processing steps predicted as necessary to produce the given viable target molecule in that host organism. As another example, the prediction engine 109 may select a host organism based upon the predicted yield of the viable target molecule produced by that host organism. Predicted yield may be derived in a number of ways, including Flux-Balance Analysis (FBA) based on a separate model for each potential host, simple elemental yield modeling, and precursor-based percent yield estimates. The prediction engine 109 provides, as output, data representing the host organisms determined to satisfy the at least one criterion.

As described for embodiments above, the prediction engine 109 may generate a record of one or more reaction pathways (i.e., pedigrees) leading to each target molecule produced by each host organism. Based on the above embodiments that run the tool for the plurality of host organisms, the reaction annotation engine 107 may store associations between host organisms, target molecules, and pedigrees in a database as a library, which may include annotations specifying parameters such as yield, number of processing steps, availability of catalysts to catalyze reactions in the reaction pathways, etc. Alternatively, the library may be obtained from a third party.

In embodiments, if the prediction engine 109 has access to such a library, the tool need not be run to identify multiple host organisms in which to produce a given viable target molecule. Instead, in such embodiments, the prediction engine 109 may use the pedigrees from the library, which may include annotation data concerning associations among the hosts, target molecules, and reactions. The prediction engine 109 may identify at least one target host organism from among the one or more host organisms based at least in part upon evidence, from, e.g., the library or public or proprietary databases, that all the catalysts predicted to catalyze reactions in at least one reaction pathway leading to production of the target molecule in the at least one target host organism are likely available to catalyze all such reactions in the at least one reaction pathway. In embodiments, the prediction engine 109 may determine target hosts based upon the target hosts requiring less than a threshold number of reaction steps within the reaction pathways that are predicted as necessary to produce the target molecule.

Bioprospecting. Some reaction enzymes may have an EC number and be well-characterized (their reactants and products are known), but not have a known associated amino acid sequence or genetic sequence (“orphan enzymes”). In such cases, the prediction engine 109 may bioprospect the orphan enzymes to predict their amino acid sequences, and, ultimately, their genetic sequences, so that the newly-sequenced enzymes may be engineered into the host organism to catalyze one or more reactions. The prediction engine 109 may then designate the reactions corresponding to the newly-sequenced enzymes as members of the filtered reaction data. In embodiments, the prediction engine 109 bioprospects the orphan enzymes using techniques known in the art. For example, one team determined the amino acid sequences for a small number of orphan enzymes by applying mass-spectrometry based analysis and computational methods (including sequence similarity networks and operon context analysis) to identify sequences. The team then used the newly determined sequences to more accurately predict the catalytic function of many more previously uncharacterized or misannotated proteins. Ramkissoon K R, et al. (2013) Rapid Identification of Sequences for Orphan Enzymes to Power Accurate Protein Annotation, PLoS ONE 8(12): e84508. doi: 10. 1371/journal.pone.0084508; see also Shearer A G, et al. (2014) Finding Sequences for over 270 Orphan Enzymes. PLoS ONE 9(5): e97250. doi:10.1371/journal.pone.0097250; Yamada T, et al., Prediction and identification of sequences coding for orphan enzymes using genomic and metagenomic neighbours genomic and metagenomic neighbours, Molecular Systems Biology 8:581, all three of which are incorporated by reference in their entirety herein.

Genome engineering. The bioreachable prediction tool may provide the list of bioreachable candidate molecules (viable target molecules) to a chemist, materials scientist or the like, who may be a third party such as a customer. Based upon their choice of target molecules, the user may instruct the tool to provide, to a gene manufacturing system, indications of the genetic sequences for the enzymes or other catalysts used to catalyze the reactions in the reaction pathways leading to each selected target molecule. The gene manufacturing system may then embody (through, e.g., insertion, replacement, deletion) the indicated genetic sequences into the genome of the host, to thereby produce an engineered genome for manufacture of the viable target molecules. In embodiments, the gene manufacturing system may be implemented using by systems and techniques known in the art, or by the factory 210 described in pending U.S. patent application, Ser. No. 15/140,296, filed Apr. 27, 2016, entitled “Microbial Strain Design System and Methods for Improved Large Scale Production of Engineered Nucleotide Sequences,” incorporated by reference in its entirety herein. In embodiments, the prediction engine 109 provides to the factory an indication of one or more catalysts for the factory to introduce the one or more catalysts into the growth medium of the host organism for production of the target molecule.

### Pathway Prediction Examples

The prediction engine 109 may predict every pathway of reactions employing catalysts likely available to be catalyzed or engineered to reach a target molecule, according to embodiments of the disclosure. The prediction engine 109 may also be used to select from among the predicted pathways to attempt manufacturing of the molecule based on qualitative information or quantitative information such as a score that may be generated by the prediction engine 109.

### Reaction Labels and Categories

Reaction sets can be filtered and labeled as described elsewhere in this patent. For example, reactions can be labeled as “sequence relaxed,” to indicate they are likely to have gene sequences available, or they could be labeled as “characterized orphan” to indicate that genes exist in nature, but need to be experimentally characterized. Reactions can similarly be labeled to reflect their mass and energy balance, or other traits.

In addition, the BPT may calculate in which direction a reaction is likely to operate based on thermodynamic data.

During processing of reactions to generate target molecules, the reaction annotation engine 107 can flag whether the production of a target molecule by a reaction happens in the thermodyanamically favorable direction or in the thermodynamically unfavorable direction.

These thermodynamic results and all of the other reaction labels can then be used by the reaction annotation engine 107 to tag the molecules and pedigrees produced by a given run of the BPT. For example, a five-step pedigree that contains one thermodynamically unfavorable reaction and two reactions lacking known genes to produce enzymes to catalyze the reactions could be labeled as:

Path length: 5

Unfavorable reactions: 1

Reactions lacking genes: 2

These labels then may be used by the prediction engine 109 to score each reaction. They also can be used to sort and operate on subsections of output, and they provide a direct insight into the engineerability of a given molecule for a given host.

In the examples detailed below, the BPT was used to identify bioreachable target molecules and display predicted pathways that may be used to reach those target molecules.

Thermodynamic data that was incorporated into pathway production and evaluation was generated using the group contribution method, but could also have been derived from any number of metabolic databases.

The prediction engine 108 may assign to each potential pathway an associated score created using the scoring method described herein. These scores can be used to inform decisions about which pathway variation to attempt to engineer to make the target molecule.

In embodiments, the prediction engine 109 may start with an optimal score of 100 points and subtract points for pathway features that add difficulty or risk of design failure. For example, path length correlates with design risk, and the total score may be reduced as path length increases, e.g., the prediction engine 109 may subtract from the score one or more points for each additional step in path length.

### Tyramine

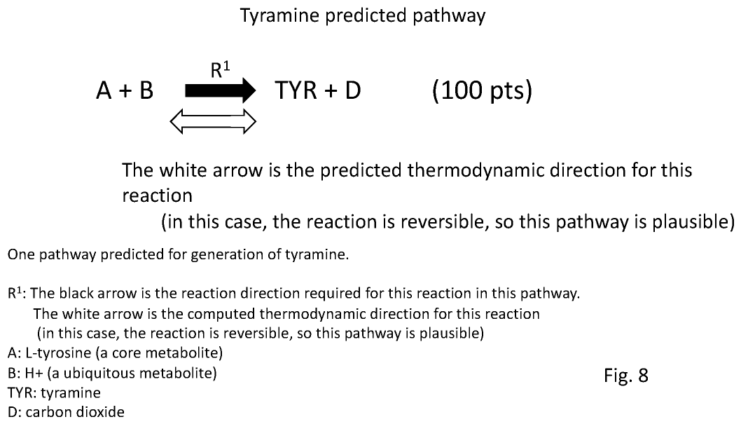

FIG. 8 illustrates a pathway identified by the prediction engine 109 to produce tyramine, according to embodiments of the disclosure. In the case of tyramine, a single pathway consisting of one reaction step (R¹) was predicted. The pathway shown depends on a reaction that is calculated based on thermodynamic data to be reversible, meaning it can operate in the direction required to generate tyramine.

In the pathway figures, a black arrow represents the reaction direction required for that reaction in the pathway to produce the desired molecule (here, tyramine). A white arrow represents the calculated thermodynamic direction for a reaction. When the required and calculated reaction directions match, the pathway is plausible.

This single pathway scores 100 points by the metric described elsewhere. (S)-2,3,4,5-tetrahydrodipicolinate (THDP)

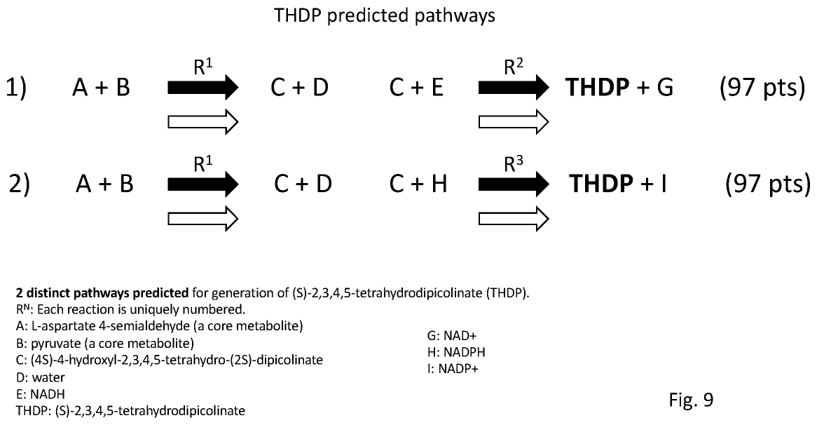

As shown in FIG. 9, the BPT predicted two possible two-step pathways to generate THDP, according to embodiments of the disclosure. Both pathways achieve the same score of 97 points in these embodiments.

The pathways share the same first reaction (R¹) and differ at the second reaction (R² or R³). In this case, these reactions differ in which form of reducing cofactor they use, e.g., NADH versus NADPH. Although the pathways score the same, this cofactor difference is relevant for engineering purposes, and thus is displayed in this embodiment of the BPT to help guide design decisions. Typically, one cofactor (either NADH or NADPH) is far more abundantly present in each given host organism. Thus, in embodiments, one skilled in the art may select the pathway employing the more abundant cofactor to produce THDP. In other embodiments, the prediction engine 109 may retrieve from a database and consider information concerning the influence of cofactors on engineerability to compute the target molecule score, thereby obviating the need for human review of the pathway cofactors.

Example predicted pathways for hypothetical molecule “F”

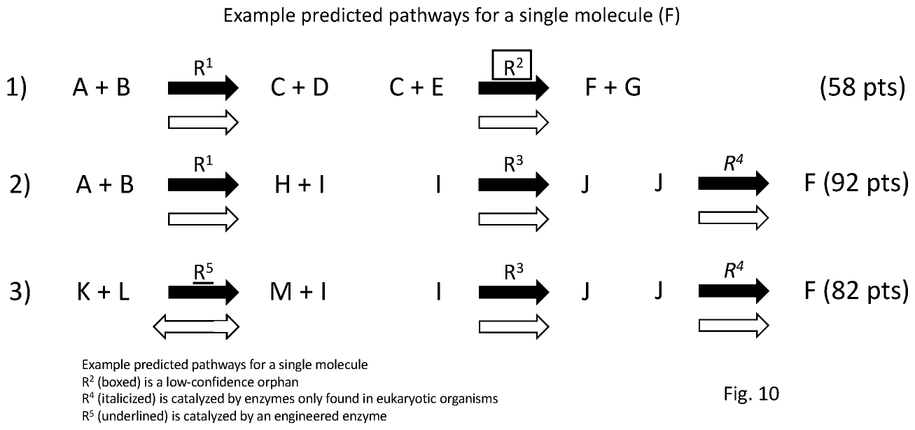

In another example, for a bioreachable molecule “F,” the BPT has predicted three potential pathways, as illustrated in FIG. 10.

The first pathway is two steps long and includes a low-confidence orphan reaction (R²), leading to a score of 58 points. A low-confidence orphan reaction is a reaction catalyzed by an orphan enzyme for which it is unlikely that the corresponding DNA sequence is readily available without extensive, specific research work. Thus, many points are deducted for the orphan enzyme.

The second pathway is three steps long and includes one reaction with only eukaryotic genes available (R⁴), leading to a score of 92 points. Points are deducted because of overall pathway length and because of the limitation in sourcing genes for R⁴.

The third pathway is also three steps long and has two reactions (R³ and R⁴) in common with the other three-step reaction. It also has one reaction (R⁴) with only eukaryotic genes available and another reaction (R⁵) that requires an engineered enzyme, leading to a score of 82 points. In addition, this pathway has an alternate set of starting core metabolites (K+L instead of A+B) which has no impact on the pathway score, but is a consideration when deciding on which pathway is a best fit for the specific host and application.

In this example, the scoring output from the BPT's prediction engine 109 provides critical engineering information beyond simple path length. Despite intuition that the shortest pathway (#1) might be best, information collected by the annotation engine 107 about each reaction and by the BPT during filtering or processing show that the longer pathways (#2 and #3) might be more feasible to engineer. For example, the reaction annotation engine 107 may determine that catalysts for some reactions are only available in high-risk categories (e.g. low-confidence orphans, engineered enzymes), and the prediction engine 109 may determine that the short pathway depends on these high-risk categories whereas the long pathway does not, which may show that a longer pathway may be more feasible to engineer.

### Tetrahydrodipicolinate Scoring Table

According to embodiments of the disclosure, the prediction engine 109 uses the information it generates to score the difficulty of producing target molecules. (Conversely, the score may be viewed as indicating the ease of producing molecules.) This score is interchangeably referred to herein as “molecule score,” “target molecule score,” or “overall pathway score.”

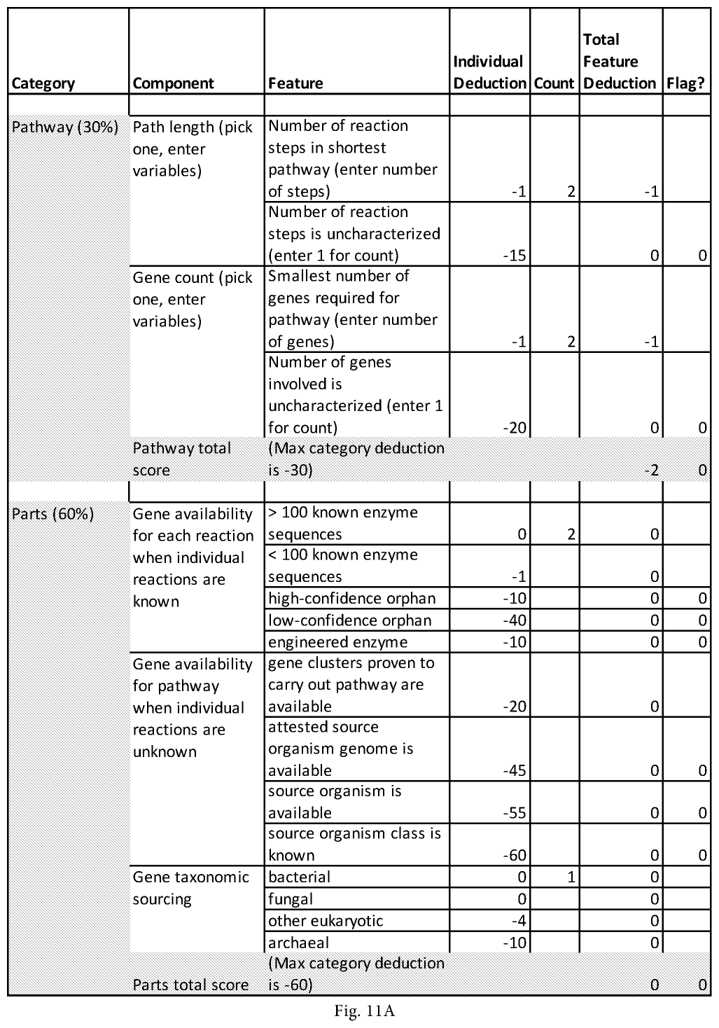

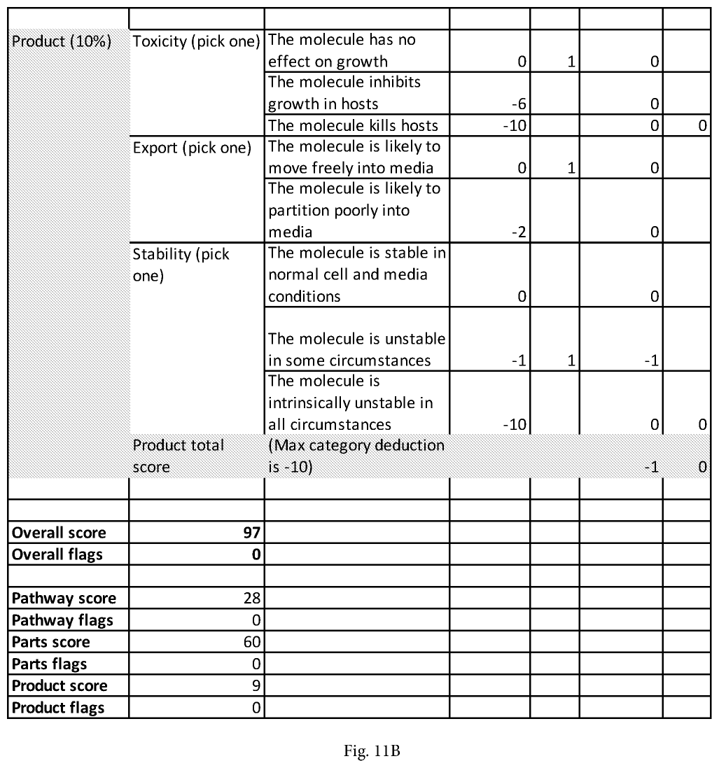

As an example, FIGS. 11A and 11B together provide a table illustrating how the prediction engine 109 may score the production of tetrahydrodipicolinate (TFIDP). In embodiments, the overall pathway scoring process may be broken down by components such as pathway score, parts score, and product score, weighted, e.g., as 30%, 60%, 10%, as shown in the table. The evaluation data shown was generated during the process of predicting pathways to the molecule (S)-2,3,4,5-tetrahydrodipicolinate (TUDP).

Pathway component score represents the relative engineering feasibility of the pathway. In embodiments, it comprises two elements:

Path length—The number of reaction steps in the pathway. This is tallied as an intrinsic part of bioreachable prediction by the prediction engine 109, according to embodiments of the disclosure.

Gene count—The number of genes predicted to be required for the pathway. This is identified by querying databases as part of reaction filtering by the reaction annotation engine 107.

Since reactions and enzymes are not always in a 1:1 relationship (for example, a single reaction is sometimes catalyzed by a two-part enzyme, requiring two genes), the prediction engine 109 may factor both elements into the predicted difficulty of engineering the pathway.

In both pedigrees predicted by the BPT, as shown in FIG. 9 THDP requires a two-step pathway in the desired host organism. This yields an appropriate score deduction based on the modest increase in difficulty of a 2- versus 1-step pathway.

In this case, the number of genes per pathway reaction step (identifiable via the same evaluation process that determines if a reaction is likely to have genes at all) also yields a modest penalty.

### Parts Component Score

The Parts score represents the relative engineering feasibility of the individual pathway parts. In embodiments, it is based on the predicted difficulty in finding the parts (e.g., genes) required to engineer a catalyst into a host for the reactions in the pathway that is being evaluated.

In embodiments, the possible features that can impact the ability to find parts include:

>100 known enzyme sequences—100 or more sequences found for the reaction during the reaction filtering step (e.g., 100 or more amino acid sequences indicated in at least one database corresponding to enzymes for catalyzing the reaction)

<100 known enzyme sequences—enzyme sequences were found, but fewer than 100 were identified during the reaction filtering step

high-confidence orphan/low-confidence orphan—no enzyme sequences were found in public databases during the reaction filtering step, but associated evidence was found that suggested those sequences would be relatively easy (high-confidence) or hard (low-confidence) to identify

engineered enzyme—the only enzymes linked to this reaction during the reaction filtering step were engineered to carry out the reaction (this data can be found in database searches). This typically refers to natural enzymes that have been mutated to catalyze a reaction different from the reaction they naturally catalyze. These engineered enzymes can be difficult to use in novel pathways as they may be limited to one or a few sequences from a limited range of donor organisms. Such engineered enzymes can be found in public databases such as BRENDA

gene taxonomic sourcing—also identified during the reaction filtering step (assuming enzyme sequences were found); this component classifies a bioreachable molecule by the “worst case” (biggest penalty) among the reactions in the predicted pathway for that bioreachable molecule; penalties are based on empirical data to date on the difficulty of expressing enzymes from the indicated sources in industrial platform organisms

gene availability for pathway when individual reactions are unknown—in some cases pathways are defined using stand-in reactions in the dataset, and these reactions can be programmatically linked to individual gene clusters or organisms; pathways in which individual reactions are unknown represent a significant increase in engineering risk and difficulty and thus a large penalty is assigned

These feature elements are all identified by the reaction annotation engine 107, as information is accumulated about the presence, absence, and abundance of sequence data for enzymes that catalyze each reaction.

In the case of THDP, genes are abundantly present for both pathway reactions, yielding no penalty. If instead, for example, one of the reactions were catalyzed by a low-confidence orphan, THDP would have accrued a significant penalty.

### Product Component Score

The Product score is the smallest overall contributor to the target molecule score, in embodiments of the disclosure. The product score represents factors that influence the difficulty in sustaining the product in the cell, exporting it from the cell, and maintaining it in media. In embodiments, it represents an evaluation of the molecule's expected toxicity, exportability, and stability. The specific features described in this embodiment include:

Toxicity—The degree to which the molecule might be expected to be toxic to one or more host organisms. This information can be derived from querying antimicrobial databases (or other databases that collect toxicity information on the general category of host organisms).

Export—Predicted by querying chemical databases for partition coefficient data, or by querying internal experimental data.

Stability—Stability issues are identified by querying chemical databases.

### Score Summary

The bottom of the table summarizes the overall score and the category scores. It also highlights any flags—areas that require particular derisking for pathway engineering. THDP happens to have no flags. An example flag would be if a pathway is missing one or more genes for its reaction steps (e.g., high- or low-confidence orphans).

### Computer System Implementation

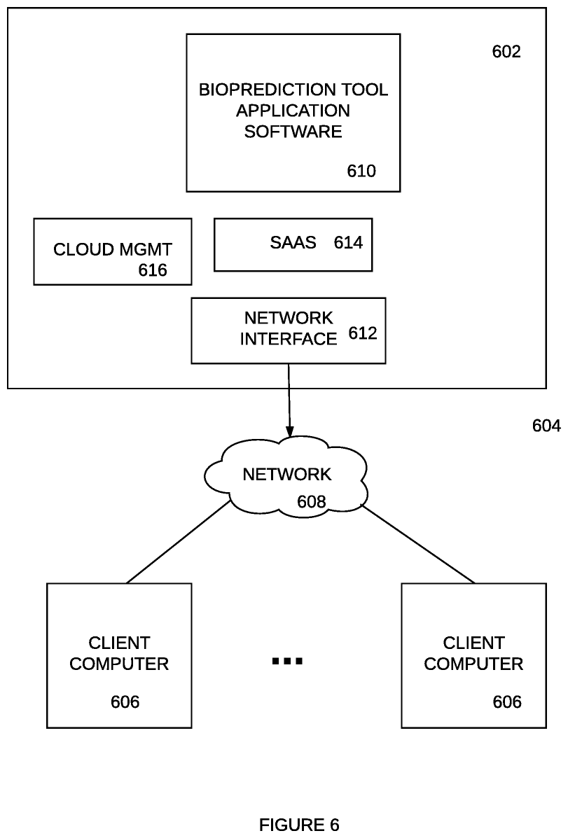

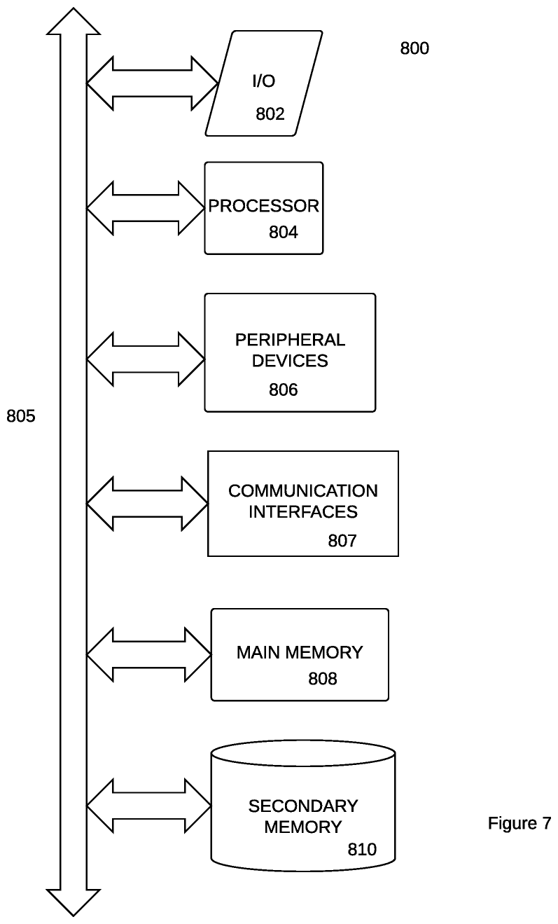

FIG. 6 illustrates a cloud computing environment 604 according to embodiments of the present disclosure. In embodiments of the disclosure, the software 610 for the reaction annotation engine 107 and the prediction engine 109 of FIG. 1 may be implemented in a cloud computing system 602, to enable multiple users to annotate reactions and predict bioreachable molecules according to embodiments of the present disclosure. Client computers 606, such as those illustrated in FIG. 7, access the system via a network 608, such as the Internet. The system may employ one or more computing systems using one or more processors, of the type illustrated in FIG. 7. The cloud computing system itself includes a network interface 612 to interface the bioreachable prediction tool software 610 to the client computers 606 via the network 608. The network interface 612 may include an application programming interface (API) to enable client applications at the client computers 606 to access the system software 610. In particular, through the API, client computers 606 may access the annotation engine 107 and the prediction engine 109.

A software as a service (SaaS) software module 614 offers the BPT system software 610 as a service to the client computers 606. A cloud management module 616 manages access to the system 610 by the client computers 606. The cloud management module 616 may enable a cloud architecture that employs multitenant applications, virtualization or other architectures known in the art to serve multiple users.

FIG. 7 illustrates an example of a computer system 800 that may be used to execute program code stored in a non-transitory computer readable medium (e.g., memory) in accordance with embodiments of the disclosure. The computer system includes an input/output subsystem 802, which may be used to interface with human users and/or other computer systems depending upon the application. The I/O subsystem 802 may include, e.g., a keyboard, mouse, graphical user interface, touchscreen, or other interfaces for input, and, e.g., an LED or other flat screen display, or other interfaces for output, including application program interfaces (APIs). Other elements of embodiments of the disclosure, such as the annotation engine 107 and the prediction engine 109, may be implemented with a computer system like that of computer system 800.

Program code may be stored in non-transitory media such as persistent storage in secondary memory 810 or main memory 808 or both. Main memory 808 may include volatile memory such as random access memory (RAM) or non-volatile memory such as read only memory (ROM), as well as different levels of cache memory for faster access to instructions and data. Secondary memory may include persistent storage such as solid state drives, hard disk drives or optical disks. One or more processors 804 reads program code from one or more non-transitory media and executes the code to enable the computer system to accomplish the methods performed by the embodiments herein. Those skilled in the art will understand that the processor(s) may ingest source code, and interpret or compile the source code into machine code that is understandable at the hardware gate level of the processor(s) 804. The processor(s) 804 may include graphics processing units (GPUs) for handling computationally intensive tasks.

The processor(s) 804 may communicate with external networks via one or more communications interfaces 807, such as a network interface card, WiFi transceiver, etc. A bus 805 communicatively couples the I/O subsystem 802, the processor(s) 804, peripheral devices 806, communications interfaces 807, memory 808, and persistent storage 810. Embodiments of the disclosure are not limited to this representative architecture. Alternative embodiments may employ different arrangements and types of components, e.g., separate buses for input-output components and memory subsystems.

Those skilled in the art will understand that some or all of the elements of embodiments of the disclosure, and their accompanying operations, may be implemented wholly or partially by one or more computer systems including one or more processors and one or more memory systems like those of computer system 800. In particular, the elements of bioreachable prediction tool and any other automated systems or devices described herein may be computer-implemented. Some elements and functionality may be implemented locally and others may be implemented in a distributed fashion over a network through different servers, e.g., in client-server fashion, for example. In particular, server-side operations may be made available to multiple clients in a software as a service (SaaS) fashion, as shown in FIG. 6.

Although the disclosure may not expressly disclose that some embodiments or features described herein may be combined with other embodiments or features described herein, this disclosure should be read to describe any such combinations that would be practicable by one of ordinary skill in the art.

Those skilled in the art will recognize that, in some embodiments, some of the operations described herein may be performed by human implementation, or through a combination of automated and manual means. When an operation is not fully automated, appropriate components of bioreachable prediction tool may, for example, receive the results of human performance of the operations rather than generate results through its own operational capabilities.

1-104. (canceled)

105. A computer-implemented method for predicting the viability of producing target molecules in a host organism, the method comprising:
using at least one processor, selecting reactions based at least in part upon whether the reactions are indicated as catalyzed by one or more corresponding catalysts that are themselves indicated as available to catalyze the reactions, wherein a reaction set comprises the selected reactions;
in each processing step of one or more processing steps performed by at least one processor, processing, pursuant to one or more reactions in the reaction set, data representing starting metabolites for the host organism and metabolites generated in previous processing steps, to generate data representing one or more predicted viable target molecules; and
using at least one processor, providing, as output, data representing the one or more predicted viable target molecules.

106. The method of claim 105, wherein selecting comprises selecting reactions that are indicated as catalyzed by one or more corresponding catalysts that are themselves indicated as able to be engineered into an organism or taken up from the growth medium in which an organism is grown.

107. The method of claim 105, wherein selecting comprises selecting reactions that are indicated as catalyzed by one or more corresponding catalysts that are themselves indicated as corresponding to one or more amino acid sequences or one or more genetic sequences.

108. The method of claim 105, wherein selecting comprises selecting reactions based at least in part upon whether the reactions are indicated in at least one database as catalyzed by one or more corresponding catalysts that are themselves indicated as available to catalyze the reactions.

109. The method of claim 105, wherein the one or more corresponding catalysts comprise an enzyme or an enzyme-nanoparticle conjugate.

110. The method of claim 105, wherein selecting reactions comprises selecting at least one reaction based at least in part upon a degree of confidence as to whether a catalyst is available to catalyze the at least one reaction in the host organism.

111. The method of claim 105, wherein selecting comprises selecting reactions that are indicated as spontaneous or indicated as catalyzed by an orphan enzyme.

112. The method of claim 105, further comprising generating a record of one or more reaction pathways leading to a predicted viable target molecule.

113. The method of claim 105, further comprising generating an indication of the difficulty of producing one or more of the predicted viable target molecules.

114. The method of claim 105, further comprising providing to a gene manufacturing system an indication of one or more genetic sequences associated with one or more reactions in a reaction pathway leading to a predicted viable target molecule, wherein the gene manufacturing system is operable to embody the indicated one or more genetic sequences into the genome of the host organism, to produce an engineered genome for manufacture of the predicted viable target molecule.

115. A system for predicting the viability of producing target molecules in a host organism, the system comprising:
one or more processors;
one or more memories operatively coupled to the one or more processors and comprising instructions, that when executed by at least one of the one or more processors, cause the system to:
select reactions based at least in part upon whether the reactions are indicated as catalyzed by one or more corresponding catalysts that are themselves indicated as available to catalyze the reactions, wherein a reaction set comprises the selected reactions;
in each processing step of one or more processing steps performed by at least one of the one or more processors, processing, pursuant to one or more reactions in the reaction set, data representing starting metabolites for the host organism and metabolites generated in previous processing steps, to generate data representing one or more predicted viable target molecules; and
providing, as output, data representing the one or more predicted viable target molecules.

116. The system of claim 115, wherein selecting comprises selecting reactions that are indicated as catalyzed by one or more corresponding catalysts that are themselves indicated as able to be engineered into an organism or taken up from the growth medium in which an organism is grown.

117. The system of claim 115, wherein selecting comprises selecting reactions that are indicated as catalyzed by one or more corresponding catalysts that are themselves indicated as corresponding to one or more amino acid sequences or one or more genetic sequences.

118. The system of claim 115, wherein selecting comprises selecting reactions based at least in part upon whether the reactions are indicated in at least one database as catalyzed by one or more corresponding catalysts that are themselves indicated as available to catalyze the reactions.

119. The system of claim 115, wherein the one or more corresponding catalysts comprise an enzyme or an enzyme-nanoparticle conjugate.

120. The system of claim 115, wherein selecting reactions comprises selecting at least one reaction based at least in part upon a degree of confidence as to whether a catalyst is available to catalyze the at least one reaction in the host organism.

121. The system of claim 115, wherein selecting comprises selecting reactions that are indicated as spontaneous or indicated as catalyzed by an orphan enzyme.

122. The system of claim 115, the one or more memories further comprising instructions for generating a record of one or more reaction pathways leading to a predicted viable target molecule.

123. The system of claim 115, the one or more memories further comprising instructions for providing to a gene manufacturing system an indication of one or more genetic sequences associated with one or more reactions in a reaction pathway leading to a predicted viable target molecule, wherein the gene manufacturing system is operable to embody the indicated one or more genetic sequences into the genome of the host organism, to produce an engineered genome for manufacture of the predicted viable target molecule.

124. One or more non-transitory computer-readable media storing instructions for predicting the viability of producing target molecules in a host organism, wherein the instructions, when executed by one or more computing devices, cause at least one of the one or more computing devices to:
select reactions based at least in part upon whether the reactions are indicated as catalyzed by one or more corresponding catalysts that are themselves indicated as available to catalyze the reactions, wherein a reaction set comprises the selected reactions;
in each processing step of one or more processing steps performed by at least one of the one or more computing devices, processing, pursuant to one or more reactions in the reaction set, data representing starting metabolites for the host organism and metabolites generated in previous processing steps, to generate data representing one or more predicted viable target molecules; and
providing, as output, data representing the one or more predicted viable target molecules.

125. The one or more non-transitory computer-readable media of claim 124, wherein selecting comprises selecting reactions that are indicated as catalyzed by one or more corresponding catalysts that are themselves indicated as able to be engineered into an organism or taken up from the growth medium in which an organism is grown.

126. The one or more non-transitory computer-readable media of claim 124, wherein selecting comprises selecting reactions that are indicated as catalyzed by one or more corresponding catalysts that are themselves indicated as corresponding to one or more amino acid sequences or one or more genetic sequences.

127. The one or more non-transitory computer-readable media of claim 124, wherein selecting comprises selecting reactions based at least in part upon whether the reactions are indicated in at least one database as catalyzed by one or more corresponding catalysts that are themselves indicated as available to catalyze the reactions.

128. The one or more non-transitory computer-readable media of claim 124, wherein the one or more corresponding catalysts comprise an enzyme or an enzyme-nanoparticle conjugate.

129. The one or more non-transitory computer-readable media of claim 124, wherein selecting reactions comprises selecting at least one reaction based at least in part upon a degree of confidence as to whether a catalyst is available to catalyze the at least one reaction in the host organism.

130. The one or more non-transitory computer-readable media of claim 124, wherein selecting comprises selecting reactions that are indicated as spontaneous or indicated as catalyzed by an orphan enzyme.

131. The one or more non-transitory computer-readable media of claim 124 further storing instructions that, when executed by one or more computing devices, cause at least one of the one or more computing devices to generate a record of one or more reaction pathways leading to a predicted viable target molecule.

132. The one or more non-transitory computer-readable media of claim 124 further storing instructions that, when executed by one or more computing devices, cause at least one of the one or more computing devices to generate an indication of the difficulty of producing one or more of the predicted viable target molecules.

133. The one or more non-transitory computer-readable media of claim 124 further storing instructions that, when executed by one or more computing devices, cause at least one of the one or more computing devices to provide to a gene manufacturing system an indication of one or more genetic sequences associated with one or more reactions in a reaction pathway leading to a predicted viable target molecule, wherein the gene manufacturing system is operable to embody the indicated one or more genetic sequences into the genome of the host organism, to produce an engineered genome for manufacture of the predicted viable target molecule.

134. One or more non-transitory computer-readable media storing instructions for identifying one or more host organisms in which to produce a target molecule, wherein the instructions, when executed by one or more computing devices, cause at least one of the one or more computing devices to:
select at least one of the one or more host organisms as one or more target host organisms in which to produce a target molecule based at least in part on evidence that one or more catalysts involved in producing the target molecule can be engineered into the one or more target host organisms or taken up from the growth medium in which the one or more target host organisms are grown; and
provide, as output, data representing the one or more target host organisms.
